(19) United States
(12) Patent Application Publication
(10) Pub. No.: US 2026/0148857 A1
(43) Pub. Date: May 28, 2026

(54) APPARATUS AND METHOD USING LIGHTWEIGHTED TRANSFORMER MODEL FOR HUMAN ACTIVITY RECOGNITION IN PORTABLE DEVICES

(71) Applicant: PUSAN NATIONAL UNIVERSITY INDUSTRY-UNIVERSITY COOPERATION FOUNDATION, Busan (KR)

(72) Inventors: Yunju BAEK, Busan (KR); Taegu KIM, Busan (KR); Hyesun JEONG, Busan (KR)

(73) Assignee: PUSAN NATIONAL UNIVERSITY INDUSTRY-UNIVERSITY COOPERATION FOUNDATION, Busan (KR)

(21) Appl. No.: 19/243,715

(22) Filed: Jun. 20, 2025

(30) Foreign Application Priority Data
Nov. 26, 2024 (KR) 10-2024-0171182

(51) Int. Cl. G16H 50/30

(52) U.S. Cl. CPC G16H 50/30

(57) ABSTRACT

An apparatus using a lightweight transformer model for human activity recognition in a portable device, includes: a data collector configured to collect data for human activity recognition based on an mmWave radar; a data processor configured to perform a data processing process to process mmWave data and convert the mmWave data into an input form for a model; a lightweight GST model part configured to generate a feature vector by combining a grouped attention mechanism, which splits an input sequence into several small groups and independently calculates attention within each group, and a sparse attention mechanism, which calculates attention only for selected location pairs rather than calculating attention for all location pairs, to classify an output class through a fully connected layer; and a human activity recognition result outputter configured to output human activity recognition results based on human activity types classified in the lightweight GST model part.

### CROSS-REFERENCE TO RELATED PATENT APPLICATION

This application claims priority to Korean Patent Application No. 10-2024-0171182 (filed on Nov. 26, 2024), which is hereby incorporated reference in its entirety.

### ACKNOWLEDGEMENT

This work was supported by the Institute of Information & Communications Technology Planning & Evaluation (IITP), Republic of Korea under the Artificial Intelligence Convergence Innovation Human Resources Development (IITP-2025-RS-2023-00254177) grant funded by the Ministry of Science and ICT (MSIT), Republic of Korea.

### BACKGROUND

The present disclosure relates to human activity recognition, and more specifically, to an apparatus and a method using a lightweight transformer model (grouped sparse transformer, GST) for human activity recognition in portable devices, which enable real-time human activity recognition in portable devices based on a lightweight transformer model.

Recently, interest in healthcare and human activity monitoring technologies has been rapidly increasing due to the aging society and COVID-19 pandemic. In particular, as the elderly population increases, falls are emerging as a major health problem.

According to a 2021 report by the Korea Disease Control and Prevention Agency, 47% of the elderly population experience falls indoors, many of which result in serious injuries. These accidents are mainly caused by decreased muscle strength, medication, and loss of balance in the elderly.

In particular, falls may be life-threatening for the elderly over 65 years of age, and treatment takes a long time, so the need for a real-time monitoring system to prevent them is increasing.

In addition, early detection of abnormal behaviors that may occur in chronically ill patients and the elderly is also an important task. Vomiting, seizures, and difficulty walking may be signs of serious health conditions, and a system that monitors these physical abnormalities in real time may significantly improve the speed of medical response.

As an example, conventional camera-based monitoring systems are often affected by light or the surrounding environment, resulting in low accuracy or privacy issues. In particular, image data collected through cameras may be rejected by patients or users because of the privacy issues.

In addition, wearable devices must be directly attached to the user's body, which may hinder user convenience. The size and weight of these devices may cause inconvenience when used for long periods of time.

Specifically, human activity recognition (HAR) technology is mainly used by combining deep learning models and sensor data.

In particular, camera-based human activity recognition systems analyze user movements and actions using image and video data. These systems have become capable of very precise action recognition with the development of computer vision technology, but they have several fundamental problems.

First, they are vulnerable to environmental factors.

Conventional camera-based systems are mainly sensitive to light and lighting conditions, and their accuracy deteriorates rapidly in dark environments or at night.

This may be a major limitation considering the reality that users do not always operate in bright environments.

In addition, the camera must be in the line of sight, and if there is an object blocking the view, recognition performance drops significantly, and due to these problems, the consistency and accuracy of user monitoring are not guaranteed.

Second, there is a problem in terms of privacy protection.

Camera-based systems often transmit the user's image or video data to a server for processing, which may raise great concerns about privacy protection, and in particular, users may feel as if they are always being watched.

Many users are reluctant to use camera-based systems due to these issues.

In particular, in a medical environment, patient privacy is a very sensitive issue, so this method of data processing has practical limitations.

Third, the inconvenience of invasive sensor devices is a problem.

Conventional wearable devices or sensor-based systems require contact sensors such as inertial measurement units (IMU) to be attached to the body.

These systems may be relatively stable in terms of data collection, but they are accompanied by the inconvenience of requiring the user to wear the sensor for a long time.

In particular, if the sensor is large or heavy, it may restrict the user's activities, which may act as a factor that hinders the user experience, and the accuracy of the sensor may vary depending on the location where the sensor is attached or the user's movements.

Fourth, there is a problem of dependency on high-performance hardware.

Deep learning models require large amounts of data and high computational performance. This causes problems that are difficult to apply to low-spec hardware or portable devices.

The high computational requirements of deep learning models make it difficult to operate in real time on general mobile devices, which is a major problem in situations where real-time human activity recognition is required.

Fifth, there is difficulty in maintaining consistent performance in various environments.

Conventional technologies related to camera-based systems and sensor-based systems often perform well only in certain environments. For example, camera-based systems may show excellent performance indoors, but are affected by light and weather in outdoor environments, and sensor-based systems vary in performance depending on the user's wearing method or body movement.

This makes it difficult to meet the requirements of HAR systems that require consistent performance in various environments.

In particular, the transformer model used in the fields of natural language processing and computer vision to build HAR systems shows better performance than the existing RNN (LSTM, GRU, etc.).

The transformer model has a self-attention mechanism, and has the characteristics of each element in the input sequence learning the relationship with all other elements, diverse learning through multi-head attention parallel computation, increased computation speed, and solving long-term dependency problems.

However, the high-performance transformer model used to build the HAR system requires GPU memory, and have the problem of increased memory usage, and computational complexity that requires a large memory size and increases the amount of calculation as the number of parameters increases.

Since this is inefficient in systems where real-time response is important, model lightweighting is required.

In this way, the conventional human activity recognition technology has problems such as environmental constraints, privacy issues, invasive sensor devices, high-performance hardware requirements, and lack of consistent performance.

These problems act as major obstacles for users to consistently utilize the HAR system in real life, and a new approach such as a lightweight, non-contact transformer model is required to solve them.

Therefore, the need for a new technology that is non-contact, robust to environmental factors, and may protect the user's privacy is increasing.

### Prior-Art Documents

Korean Patent Application Publication No. 10-2023-0105868
Korean Patent Application Publication No. 10-2024-0039993
Korean Patent Application Publication No. 10-2023-0156215

### SUMMARY

The present disclosure is intended to solve the problems of the conventional human activity recognition technology, and an object is to provide an apparatus and a method using a lightweight transformer model (grouped sparse transformer, GST) for human activity recognition in portable devices, which enable real-time human activity recognition in portable devices based on a lightweight transformer model.

An object of the present disclosure is to provide an apparatus and a method using a lightweight transformer model for human activity recognition in portable devices, which enable safe monitoring of the users of their health status in a free environment and accurate human activity recognition in various environments by monitoring human activity in real time and detecting abnormal behavior such as falling or vomiting based on a non-contact multimodal sensor.

An object of the present disclosure is to provide an apparatus and a method using a lightweight transformer model for human activity recognition in portable devices, which enable efficient human activity recognition on low-spec hardware by significantly improving the computational resource requirements of a transformer model by introducing a new transformer architecture that combines grouped attention and sparse attention mechanisms.

An object of the present disclosure is to provide an apparatus and a method using a lightweight transformer model for human activity recognition in portable devices, which enable real-time human activity recognition in devices such as portable devices and wearable devices by constructing a lightweight GST model to minimize memory usage, increase computational efficiency, and optimize the model to run smoothly even on low-spec devices.

An object of the present disclosure is to provide an apparatus and a method using a lightweight transformer model for human activity recognition in portable devices, which enable resolving the privacy invasion issue of users by the recognition of activities in a non-contact manner without attaching to the user's body by utilizing an mmWave radar and various sensor data.

An object of the present disclosure is to provide an apparatus and a method using a lightweight transformer model for human activity recognition in portable devices, which enable transferring knowledge learned from a large model to a small model while maintaining the performance of a lightweight model through a knowledge distillation technique, thereby increasing computational efficiency without performance degradation.

Other objects of the present disclosure are not limited to the objects mentioned above, and other objects not mentioned will be clearly understood by those skilled in the art from the descriptions below.

An apparatus using a lightweight transformer model for human activity recognition in a portable devices according to the present disclosure to achieve the above objects includes a data collector configured to collect data for human activity recognition based on an mmWave radar; a data processor configured to perform a data processing process to process mmWave data and convert the mmWave data into an input form for a model; a lightweight GST model part configured to generate a feature vector by combining a grouped attention mechanism, which splits an input sequence into several small groups and independently calculates attention within each group, and a sparse attention mechanism, which calculates attention only for selected location pairs rather than calculating attention for all location pairs, to classify an output class through a fully connected layer; and a human activity recognition result outputter configured to output human activity recognition results based on human activity types classified in the lightweight GST model part.

Here, the grouped attention splits an input sequence into several small groups and calculates attention only within each group to reduce an amount of computation and learn local patterns, wherein multi-head attention is performed independently within each group and results of the groups are combined at the end.

In addition, the sparse attention omits interactions between unimportant tokens in a sequence and calculates attention only between selected important tokens to reduce computation on an entire sequence and improve learning performance.

In addition, the data processor includes a sliding window part configured to divide data into windows of fixed temporal size, a principal component analysis (PCA) configured to perform dimensionality reduction to reduce data complexity and retain only important features, and a data averaging part configured to average data to generate a feature vector of fixed length.

In addition, the lightweight GST model part includes a location information adder configured to add location information to input data, a data normalizer configured to normalize a distribution of the input data to stabilize learning and increase learning speed, an attention calculator configured to split the input sequence into groups and independently calculate attention for each group, a data converter configured to convert the input data through a feed-forward network (FFN), a neuron random deactivator and learning information processor configured to randomly deactivate some neurons to prevent overfitting, maintain learning information between blocks, and mitigate a gradient vanishing problem, an output value normalizer configured to normalize output values of each layer, and a feature vector generator and classifier configured to generate a final feature vector to classify an output class (human activity type) through a fully connected layer.

In addition, the attention calculator includes an input data processor configured to deliver data to next step with a sequence and location information of the data included, a data grouping processor configured to split the input data into several groups and independently calculate attention for each group, a vector calculator configured to calculate query, key, and value vectors for each group, an attention score calculator configured to calculate an attention score through a query and a key and apply a Sparse Mask to calculate relationships between necessary data, and a calculation result merger configured to merge attention calculation results for each group to generate a final output.

In addition, the query indicates what data is looking for, the key represents features of the data, and the value indicates an actual value for the key, and a sparse attention calculation calculates the attention score through the query and the key and applies the sparse mask to calculate relationships only between necessary data.

In addition, the lightweight GST model part is configured to perform knowledge distillation to transfer knowledge learned from a large model to a small model while maintaining performance of a lightweight model.

In addition, in a knowledge distillation process, a teacher model generates a soft label after recognizing the input data and delivers the soft label to a student model, and the lightweight student model recognizes the input data and learns the soft label of the teacher model and a ground truth label simultaneously, wherein learning is performed by combining distillation Loss (KL-divergence) and label loss (cross-entropy).

In addition, in a soft label generation step by the teacher model, the soft label (probability distribution) is generated through softmax based on the input data, the soft label indicates a probability predicted by the teacher model for each class, which is used to train the student model, and in a comparison step with the ground truth label, the teacher model is trained through cross-entropy loss with the ground truth label.

In addition, in a knowledge distillation process, a student model uses a soft label of a teacher model and a ground truth label for learning to optimize performance, and for the knowledge distillation, in a comparison step with the soft label, KL-divergence loss is used to train the student model to closely mimic the soft label (prediction probability distribution) of the teacher model, and in this process, knowledge of the teacher model is transferred to the student model.

In addition, in a back propagation process, the student model is optimized through back propagation based on a total loss which combines two losses (distillation loss and label loss).

A method using a lightweight transformer model for human activity recognition in a portable device according to the present disclosure to achieve another object includes performing a data processing process to process mmWave data and convert the mmWave data into an input form for a model; adding location information to input data and normalizing a distribution of the input data; splitting an input sequence into groups and independently calculating attention for each group; converting the input data through a feed-forward network (FFN) and enhancing learning expressiveness of the model; randomly deactivating some neurons to prevent overfitting, maintaining learning information between blocks, and mitigating a gradient vanishing problem; and normalizing output values of each layer and generating a final feature vector to classify an output class (human activity type) through a fully connected layer.

Here, the calculating of the attention includes delivering data to next step with a sequence and location information of the data included, splitting the input data into several groups and independently calculating attention for each group, calculating query, key, and value vectors for each group, calculating an attention score through a query and a key and applying a sparse mask to calculate relationships between necessary data, and merging attention calculation results for each group to generate a final output.

An apparatus and a method using a lightweight transformer model for human activity recognition in portable devices have the following effects.

First, real-time human activity recognition is possible in a portable device based on a lightweight transformer model (grouped sparse transformer, GST).

Second, safe monitoring of the users of their health status in a free environment and accurate human activity recognition in various environments by monitoring human activity in real time and detecting abnormal behavior such as falling or vomiting based on a non-contact multimodal sensor, is possible.

Third, efficient human activity recognition on low-spec hardware by significantly improving the computational resource requirements of a transformer model by introducing a new transformer architecture that combines grouped attention and sparse attention mechanisms, is possible.

Fourth, real-time human activity recognition in devices such as portable devices and wearable devices by constructing a lightweight GST model to minimize memory usage, increase computational efficiency, and optimize the model to run smoothly even on low-spec devices, is possible.

Fifth, resolving the privacy invasion issue of users by the recognition of activities in a non-contact manner without attaching to the user's body by utilizing an mmWave radar and various sensor data, is possible.

Sixth, transferring knowledge learned from a large model to a small model while maintaining the performance of a lightweight model through a knowledge distillation technique, thereby increasing computational efficiency without performance degradation, is possible.

### BRIEF DESCRIPTION OF THE DRAWINGS

FIG. 1 is an overall configuration diagram of an apparatus using a lightweight transformer model for human activity recognition in a portable device according to the present disclosure.

FIG. 2 is a configuration block diagram of an apparatus using a lightweight transformer model for human activity recognition in a portable device according to the present disclosure.

FIG. 3 is a detailed configuration diagram of a data processor.

FIG. 4 is a configuration diagram illustrating a data processing concept.

FIG. 5 is a detailed configuration diagram of a lightweight GST model part.

FIG. 6 is a configuration diagram illustrating an example of a lightweight GST model.

FIG. 7 is a detailed configuration diagram of an attention calculator.

FIG. 8 is a configuration diagram illustrating a knowledge distillation process.

FIG. 9 is a flow chart illustrating a method using a lightweight transformer model for human activity recognition in a portable device according to the present disclosure.

FIG. 10 is a flow chart illustrating a detailed process of attention calculation.

### DETAILED DESCRIPTION

Hereinafter, preferred embodiments of an apparatus and a method using a lightweight transformer model for human activity recognition in portable devices according to the present disclosure will be described in detail as follows.

Features and advantages of the apparatus and method using a lightweight transformer model for human activity recognition in portable devices according to the present disclosure will become apparent through the detailed description of each embodiment below.

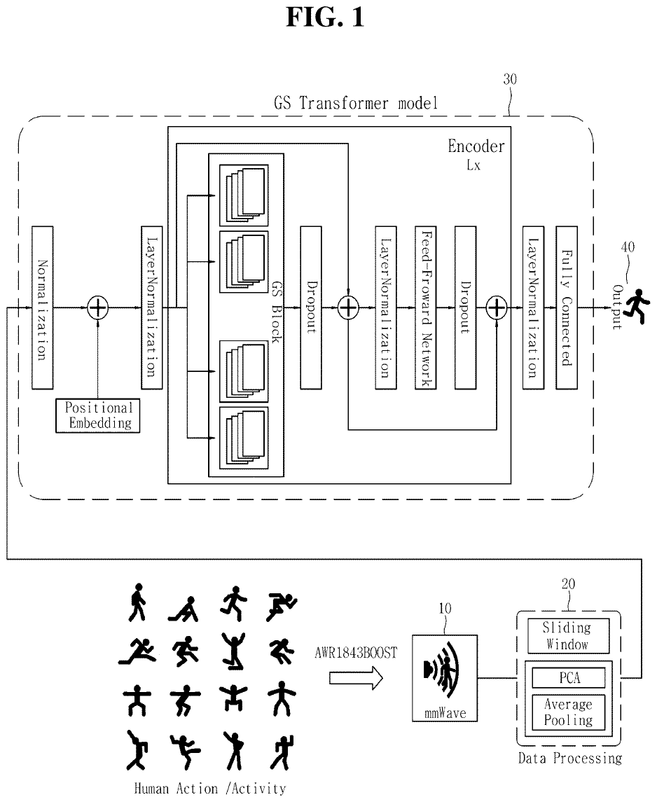

FIG. 1 is an overall configuration diagram of an apparatus using a lightweight transformer model for human activity recognition in a portable device according to the present disclosure.

The terms used in the present disclosure have been selected as general terms widely used at present as possible while considering the functions of in the present disclosure, but may vary depending on the intention of those skilled in the art, precedents, the emergence of new technologies, and the like. In addition, in certain cases, there are terms arbitrarily selected by the applicant, and in this case, their meanings will be described in detail in the relevant detailed description. Therefore, the term used in the present disclosure should be defined based on the meaning of the term and the overall content of the present disclosure, rather than simply the name of the term.

When it is said that a part “comprises” or “includes” a component throughout the specification, this means that, unless specifically stated to the contrary, the part does not exclude other components but may further include other components. In addition, the terms such as “ . . . part” and “module” used in the specification refer to a unit that processes at least one function or operation, which may be implemented as hardware or software, or as a combination of hardware and software.

In particular, units that process at least one function or operation may be implemented as an electronic device including at least one processor, and at least one peripheral device may be connected to the electronic device depending on the method of processing the function or operation. Peripheral devices may include data input devices, data output devices, and data storage devices.

The apparatus and method using a lightweight transformer model for human activity recognition in portable devices according to the present disclosure enable real-time human activity recognition in portable devices based on a lightweight transformer model (grouped sparse transformer, GST).

To this end, the present disclosure may include a configuration that enables safe monitoring of the users of their health status in a free environment and accurate human activity recognition in various environments by monitoring human activity in real time and detecting abnormal behavior such as falling or vomiting based on a non-contact multimodal sensor.

The present disclosure may include a configuration that enables efficient human activity recognition on low-spec hardware by significantly improving the computational resource requirements of a transformer model by introducing a new transformer architecture that combines grouped attention and sparse attention mechanisms.

The present disclosure may include a configuration that enables real-time human activity recognition in devices such as portable devices and wearable devices by constructing a lightweight GST model to minimize memory usage, increase computational efficiency, and optimize the model to run smoothly even on low-spec devices.

The present disclosure may include a configuration that enables resolving the privacy invasion issue of users by the recognition of activities in a non-contact manner without attaching to the user's body by utilizing an mmWave radar and various sensor data.

The present disclosure may include a configuration that enables transferring knowledge learned from a large model to a small model while maintaining the performance of a lightweight model through a knowledge distillation technique, thereby increasing computational efficiency without performance degradation.

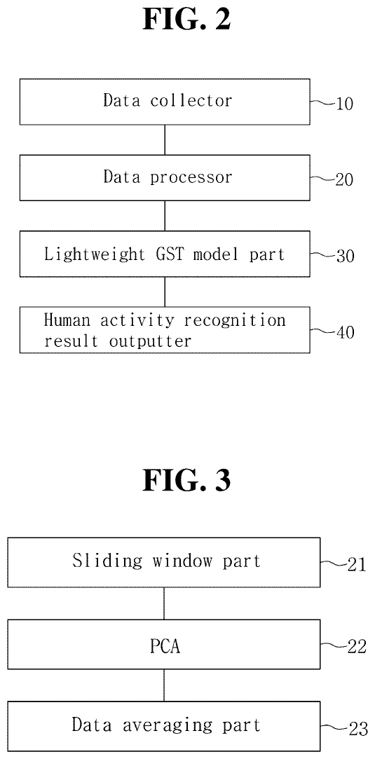

FIG. 2 is a configuration block diagram of an apparatus using a lightweight transformer model for human activity recognition in a portable device according to the present disclosure.

The apparatus using a lightweight transformer model for human activity recognition in a portable device includes, as shown in FIG. 2, a data collector 10 configured to collect data for human activity recognition based on an mmWave radar, a data processor 20 configured to perform a data processing process to process mmWave data and convert the mmWave data into a form adequate as an input for a model, a lightweight GST model part 30 configured to generate a feature vector by combining a grouped attention mechanism, which splits an input sequence into several small groups and independently calculates attention within each group, and a sparse attention mechanism, which calculates attention only for selected location pairs rather than calculating attention for all location pairs, to classify an output class through a fully connected layer, and a human activity recognition result outputter 40 configured to output human activity recognition results based on human activity types classified in the lightweight GST model part 30.

Here, the grouped attention splits an input sequence into several small groups and calculates attention only within each group to reduce the amount of calculation and to better learn local patterns.

Multi-head attention is performed independently within each group, and the results of the groups are combined at the end.

The sparse attention omits interactions between unimportant tokens within a sequence and calculates attention only between selected important tokens, allowing the model to efficiently learn important information while reducing computations for the entire sequence.

Table 1 shows an example of a data set collected based on mmWave.

TABLE 1

| Action No. | Action |
|---|---|
| A001 | Falling while walking |
| A002 | Headache |
| A003 | Chest pain |
| A004 | Stomachache |
| A005 | Back pain |
| A006 | Coughing |
| A007 | Vomiting |
| A008 | Walking |
| A009 | Running |
| A010 | Spreading arms |
| A011 | Falling forward |
| A012 | Sitting down and standing up |
| A013 | Lifting right arm and foot |
| A014 | Lifting left arm and foot |

Table 2 shows that the lightweight GST model according to the present disclosure maintains very high accuracy while having a small number of parameters and model size, and it may be confirmed that it has a higher performance than the Base Transformer at 99.78% at 30w1s.

It may be seen that the number of parameters is much smaller than the Base Transformer and the model size may be reduced.

TABLE 2

| | Window | Acc. | The number of | Model |
|---|---|---|---|---|
| Model | Size | (%) | parameters | size |
| Base | 30w1s | 74.26 | 825,499 | 1.57 MB |
| transformer | 20w1s | 61.25 | 661,659 | 1.26 MB |
| | 10w1s | 50.87 | 497,819 | 0.95 MB |
| GS-T | 30w1s | 99.78 | 308,123 | 0.59 MB |
| | 20w1s | 99.59 | 267,163 | 0.51 MB |
| | 10w1s | 97.82 | 226,203 | 0.43 MB |

Table 3 shows the performance of a lightweight GST model that combines Grouped Attention and Sparse Attention mechanisms.

TABLE 3

| | Window | Acc. | The number of | Model |
|---|---|---|---|---|
| Model | Size | (%) | parameters | size |
| Base | 30w1s | 74.26 | 825,499 | 1.57 MB |
| transformer | 20w1s | 61.25 | 661,659 | 1.26 MB |
| | 10w1s | 50.87 | 497,819 | 0.95 MB |
| Grouped | 30w1s | 99.37 | 1,248,667 | 2.38 MB |
| attention | 20w1s | 98.77 | 1,207,707 | 2.30 MB |
| | 10w1s | 94.51 | 1,207,707 | 2.30 MB |
| Sparse | 30w1s | 97.04 | 324,251 | 0.62 MB |
| attention | 20w1s | 98.24 | 283,291 | 0.54 MB |
| | 10w1s | 96.07 | 283,291 | 0.54 MB |
| GS-T | 30w1s | 99.81 | 308,123 | 0.59 MB |
| | 20w1s | 99.71 | 267,163 | 0.51 MB |
| | 10w1s | 91.44 | 226,203 | 0.43 MB |

By using grouped attention to learn local patterns more effectively, the accuracy is improved and the total number of parameters increases, and sparse attention selectively performs computations through a sparse mask to reduce the number of parameters and maintain accuracy, and it may be confirmed that the lightweight GST model combining the grouped attention and sparse attention mechanisms according to the present disclosure increases performance while reducing the amount of computation.

Grouped attention is a method of splitting a sequence into several groups and performing attention only within each group.

The input sequence is split into g groups, and multi-head attention is performed independently for each group. This reduces the amount of computation and, in particular, allows learning local patterns more effectively.

By performing attention within a group and then combining the results of all groups to create the final output, the calculation for the entire sequence length may be reduced.

In particular, grouped attention, with local pattern learning, allows better learning of local dependencies within a sequence, which is very advantageous when important information exists between close elements in long sequences, such as natural language processing or time series data.

In addition, since each group may be processed independently, parallel processing is possible, which may make the computation speed faster.

In addition, sparse attention is a method that selectively calculates only the interactions between important tokens among the entire sequence, so that important relationships may be learned without calculating the interactions between all tokens.

By using sparse mask, attention may be calculated only for selected locations and the rest may be omitted, reducing memory usage and computational complexity.

In other words, queries, keys, and values are split within each group, and only the tokens selected for each group are used for the attention computation.

The detailed configuration of the data processor 20 is as follows.

FIG. 3 is a detailed configuration diagram of a data processor.

The data processor 20 includes, as shown in FIG. 3, a sliding window part 21 configured to divide data into windows of fixed temporal size, a principal component analysis (PCA) 22 configured to perform dimensionality reduction to reduce data complexity and retain only important features, and a data averaging part 23 configured to average data to generate a feature vector of fixed length.

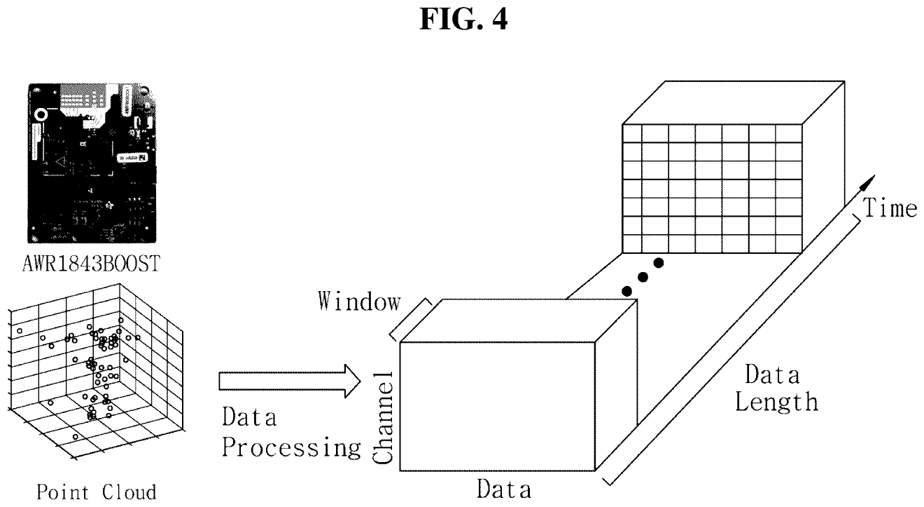

FIG. 4 is a configuration diagram illustrating a data processing concept.

A sliding window process that divides data into windows of fixed temporal size is performed.

For example, the data is split into window sizes of 1 second, 2 seconds, or 3 seconds, and it is also possible to set cases where the windows overlap, which is to maintain time order information and form an analysis unit.

In addition, the principal component analysis (PCA) process performs dimension reduction to reduce the complexity of the data and leave only important features, and this process increases the computational efficiency of the input data and removes unnecessary noise.

In addition, the average pooling process is to average the data to generate a feature vector of fixed length, thereby reducing the volatility of the input data and enabling stable learning in the next step.

The detailed configuration of the lightweight GST model part 30 is as follows.

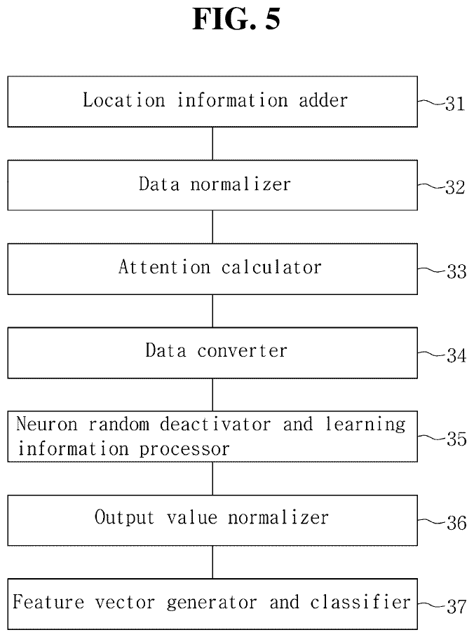

FIG. 5 is a detailed configuration diagram of a lightweight GST model part.

The lightweight GST model part 30 includes, as shown in FIG. 5, a location information adder 31 configured to add location information to input data, a data normalizer 32 configured to normalize a distribution of the input data to stabilize learning and increase learning speed, an attention calculator 33 configured to split the input sequence into groups and independently calculate attention for each group, a data converter 34 configured to convert the input data through a feed-forward network (FFN), a neuron random deactivator and learning information processor configured to randomly deactivate some neurons to prevent overfitting, maintain learning information between blocks, and mitigate a gradient vanishing problem, an output value normalizer 36 configured to normalize output values of each layer, and a feature vector generator and classifier 37 configured to generate a final feature vector to classify an output class (human activity type) through a fully connected layer.

The grouped sparse transformer (GST) model in the present disclosure performs the following operations with a lightweight transformer structure.

The positional embedding process adds location information to the input data to enable learning of order dependency. This is an important step in the transformer model, and it complements the limitations of the basic structure that does not consider order.

In addition, the normalization process normalizes the distribution of the input data to stabilize learning and improve the learning speed.

In addition, the grouped sparse attention process splits the input sequence into groups and independently calculates attention for each group.

This method reduces computational complexity and enables effective learning of local patterns. The sparse attention focuses only on necessary data and reduces memory usage.

In addition, the feed-forward network (FFN) consists of a nonlinear activation function (GELU) and two dense layers to convert input data and enhance the learning expressiveness of the model.

In addition, dropout randomly deactivates some neurons to prevent overfitting, and residual connection maintains learning information between blocks and mitigates the gradient vanishing problem even in deep networks.

In addition, the layer normalization process normalizes the output values of each layer to maintain learning stability.

In addition, global average pooling and fully connected generate the final feature vector, and classify the output class (human activity type) through the fully connected layer.

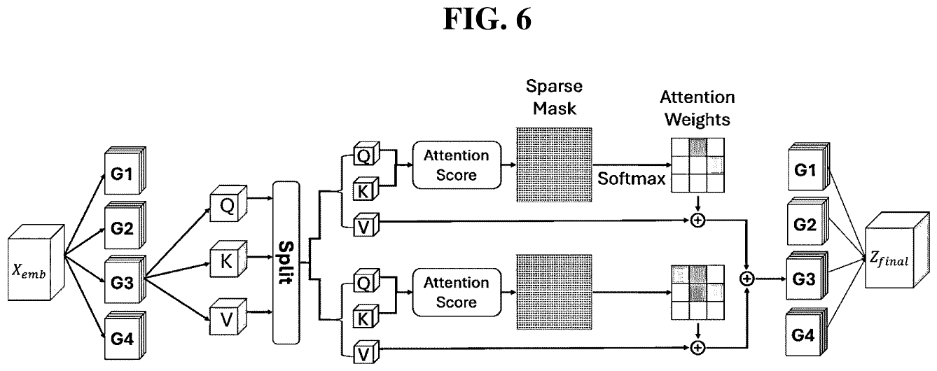

FIG. 6 is a configuration diagram illustrating an example of a lightweight GST model.

The input data (X_{emb}) block provides an embedded representation for the data input to the model, which is data generated through the positional embedding and layer normalization steps.

The data is delivered to the next step with the sequence and location information of the data included.

In addition, the grouping (G1, G2, G3, G4) block splits the input data into several groups and independently calculates attention in each group.

This groups the input data to effectively learn the local pattern of the data, and for example, splits the sequence into four groups and processes it in parallel in each group.

In addition, the query, key, value (Q, K, V) generation block calculates query, key, value vectors for each group.

Vectors are generated for extracting important information in the attention mechanism of the transformer.

Here, the query indicates what the data is looking for, the key expresses the characteristics of the data, and the value indicates the actual value for the key.

The sparse attention calculation block calculates the attention score through the query and key, and calculates only the relationship between the necessary data by applying the sparse mask.

The sparse mask performs computations only between selected locations, thereby reducing the amount of calculation and saving memory, and the calculated attention score is combined with the value to generate the final result.

In addition, the recombination (group combination) block merges the attention calculation results for each group to generate the final output.

The local patterns learned in each group are combined to supplement the global information of the entire sequence, the final output (Z_{final}) is generated.

The detailed configuration of the attention calculator 33 is as follows.

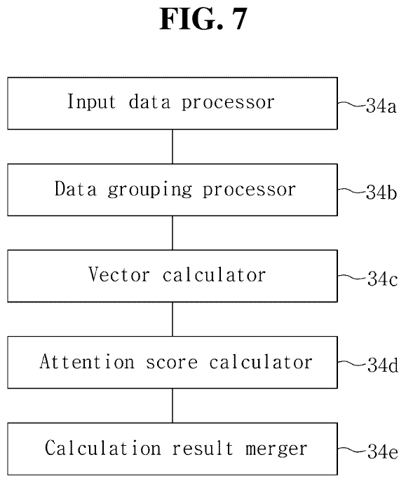

FIG. 7 is a detailed configuration diagram of an attention calculator.

The attention calculator 33 includes, as shown in FIG. 7, an input data processor 34a configured to deliver data to next step with a sequence and location information of the data included, a data grouping processor 34b configured to split the input data into several groups and independently calculate attention for each group, a vector calculator 34c configured to calculate query, key, and value vectors for each group, an attention score calculator 34d configured to calculate an attention score through a query and a key and apply a sparse mask to calculate relationships between necessary data, and a calculation result merger 34e configured to merge attention calculation results for each group to generate a final output.

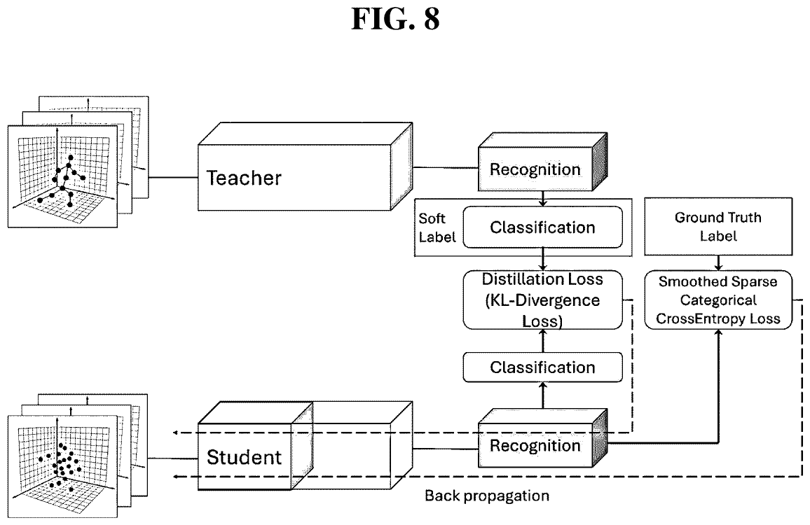

FIG. 8 is a configuration diagram illustrating a knowledge distillation process.

As shown in FIG. 8, the teacher model generates a soft label after recognizing the input data and delivers the soft label to the student model.

The lightweight student model recognizes the input data and learns the soft label of the teacher model and the ground truth label simultaneously. Learning is performed by combining distillation loss (KL-divergence) and label loss (cross-entropy).

In addition, back propagation shows the process of updating the student model based on the combined result of the two losses.

In the present disclosure, knowledge distillation may be used to transfer knowledge learned from a large model to a small model while maintaining the performance of a lightweight model.

Knowledge distillation is to transfer the knowledge of a large model (teacher model) to a lightweight small model (student model) to increase computational efficiency without performance degradation.

Specifically, the teacher model is a model that has high expressiveness and is designed with a complex and large structure and is trained with the goal of high accuracy.

The soft label generation step is to generate a soft label (probability distribution) through softmax based on input data.

The soft label represents the probability predicted by the teacher model for each class and is used to train the student model.

In the comparison step with the ground truth label, the teacher model is trained through cross-entropy loss with the ground truth label, and finally a teacher model with high performance is completed.

In addition, the student model is designed to be lightweight by simplifying the structure of the teacher model or reducing the number of parameters, and the soft label and ground truth label of the teacher model are used for learning to optimize performance.

In addition, for the knowledge distillation, in the comparison step with the soft label, KL-divergence loss is used to train the student model to closely mimic the soft label (prediction probability distribution) of the teacher model.

In this process, the knowledge (feature representation) of the teacher model is transferred to the student model.

In addition, in the comparison step with the ground truth label, the student model learns the cross-entropy loss with the actual correct label (ground truth label) to supplement the performance of the model. The smoothed sparse categorical cross-entropy loss is applied to enhance learning stability.

In addition, in the back propagation step, the student model is optimized through back propagation based on the total loss that combines the two losses (distillation loss and label loss).

Table 4 shows the lightweight characteristics through knowledge distillation, and compared to the base model, it may be confirmed that the accuracy is higher after knowledge distillation, the number of parameters is reduced compared to the teacher model, and the inference speed is increased.

TABLE 4

| | | | | | | T- | S- | | |
|---|---|---|---|---|---|---|---|---|---|
| | Sequence | T- | S- | T-Total | S-Total | Inference | Inference | T- | S- |
| Model | Length | Acc.(%) | Acc.(%) | params | params | Speed | Speed | Memory | Memory |
| Base | (Batch, 3840, 6) | 66.14 | 30.55 | 825,499 | 330,843 | — | — | — | — |
| transformer | (Batch, 640, 6) | 69.99 | 53.8 | 415,899 | 126,030 | 6.29 | 2.86 | 513.54 MB | 485.13 MB |
| | (Batch, 480, 6) | 85.59 | 79.37 | 395,419 | 115,803 | 4.09 | 1.63 | 498.95 MB | 483.84 MB |
| GSB | (Batch, 3840, 6) | 97.45 | 92.94 | 676,763 | 293,339 | — | — | — | — |
| | (Batch, 640, 6) | 98.79 | 99 | 267,163 | 88,539 | 0.85 | 0.29 | 1537.17 MB | 1525.84 MB |
| | (Batch, 480, 6) | 99.92 | 99.81 | 246,683 | 78,299 | 0.60 | 0.21 | 1534.23 MB | 1526.09 MB |

A method using a lightweight transformer model for human activity recognition in a portable device according to the present disclosure is specifically described as follows.

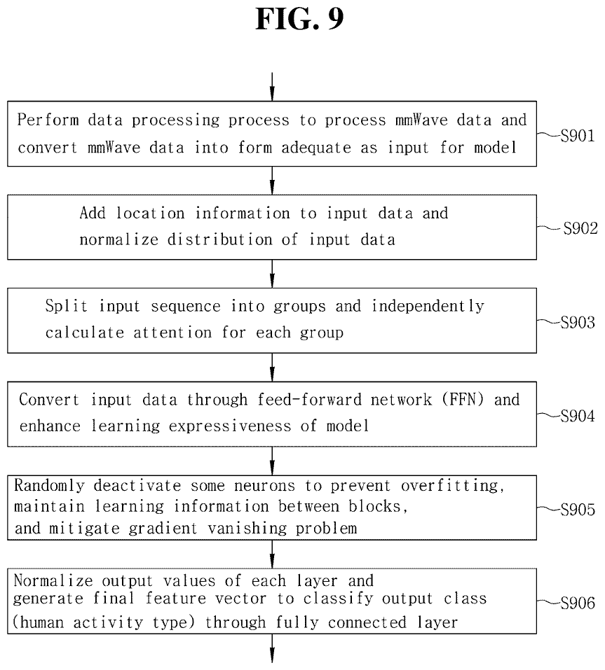

FIG. 9 is a flow chart illustrating a method using a lightweight transformer model for human activity recognition in a portable device according to the present disclosure.

The method using a lightweight transformer model for human activity recognition in a portable device according to the present disclosure includes, as shown in FIG. 9, performing a data processing process to process mmWave data and convert the mmWave data into a form adequate as an input for a model (S901), adding location information to input data and normalizing a distribution of the input data (S902), splitting an input sequence into groups and independently calculating attention for each group (S903), converting the input data through a feed-forward network (FFN) and enhancing learning expressiveness of the model (S904), randomly deactivating some neurons to prevent overfitting, maintaining learning information between blocks, and mitigating a gradient vanishing problem (S905), and normalizing output values of each layer and generating a final feature vector to classify an output class (human activity type) through a fully connected layer (S906).

The detailed process of the calculating of the attention (S903) is as follows.

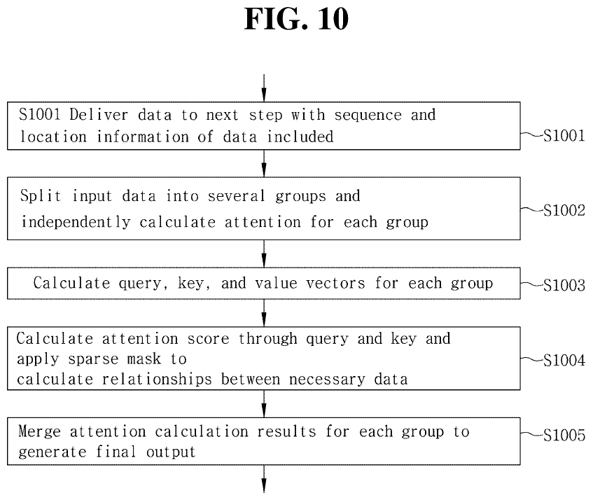

FIG. 10 is a flow chart illustrating a detailed process of attention calculation.

The calculating of the attention (S903) includes, as shown in FIG. 10, delivering data to next step with a sequence and location information of the data included (S1001), splitting the input data into several groups and independently calculating attention for each group (S1002), calculating query, key, and value vectors for each group (S1003), calculating an attention score through a query and a key and applying a sparse mask to calculate relationships between necessary data (S1004), and merging attention calculation results for each group to generate a final output (S1005).

The apparatus and method using a lightweight transformer model for human activity recognition in portable devices according to the present disclosure enables real-time human activity recognition in portable devices based on a lightweight transformer model (grouped sparse transformer, GST), and enables efficient human activity recognition on low-spec hardware by significantly improving the computational resource requirements of a transformer model by introducing a new transformer architecture that combines grouped attention and sparse attention mechanisms.

As described above, it will be understood that the present disclosure is implemented in a modified form without departing from the essential characteristics of the present disclosure.

Therefore, the specified embodiments should be considered from an illustrative rather than a restrictive perspective, and the scope of the present disclosure is indicated in the claims rather than the foregoing description, and all differences within the equivalent scope should be construed as being included in the present disclosure.

### EXPLANATION OF SYMBOLS

10. Data collector
20. Data processor
30. Lightweight GST model part
40. Human activity recognition result outputter

1. An apparatus using a lightweight transformer model for human activity recognition in a portable device, the apparatus comprising:
a data collector configured to collect data for human activity recognition based on an mmWave radar;
a data processor configured to perform a data processing process to process mmWave data and convert the mmWave data into an input form for a model;
a lightweight GST model part configured to generate a feature vector by combining a grouped attention mechanism, which splits an input sequence into several small groups and independently calculates attention within each group, and a sparse attention mechanism, which calculates attention only for selected location pairs rather than calculating attention for all location pairs, to classify an output class through a fully connected layer; and
a human activity recognition result outputter configured to output human activity recognition results based on human activity types classified in the lightweight GST model part.

2. The apparatus according to claim 1, wherein the grouped attention splits an input sequence into several small groups and calculates attention only within each group to reduce an amount of computation and learn local patterns, and
wherein multi-head attention is performed independently within each group and results of the groups are combined at the end.

3. The apparatus according to claim 1, wherein the sparse attention omits interactions between unimportant tokens in a sequence and calculates attention only between selected important tokens to reduce computation on an entire sequence and improve learning performance.

4. The apparatus according to claim 1, wherein the data processor comprises:
a sliding window part configured to divide data into windows of fixed temporal size;
a principal component analysis (PCA) configured to perform dimensionality reduction to reduce data complexity and retain only important features; and
a data averaging part configured to average data to generate a feature vector of fixed length.

5. The apparatus according to claim 1, wherein the lightweight GST model part comprises:
a location information adder configured to add location information to input data;
a data normalizer configured to normalize a distribution of the input data to stabilize learning and increase learning speed;
an attention calculator configured to split the input sequence into groups and independently calculate attention for each group;
a data converter configured to convert the input data through a feed-forward network (FFN);
a neuron random deactivator and learning information processor configured to randomly deactivate some neurons to prevent overfitting, maintain learning information between blocks, and mitigate a gradient vanishing problem;
an output value normalizer configured to normalize output values of each layer; and
a feature vector generator and classifier configured to generate a final feature vector to classify an output class (human activity type) through a fully connected layer.

6. The apparatus according to claim 5, wherein the attention calculator comprises:
an input data processor configured to deliver data to next step with a sequence and location information of the data included;
a data grouping processor configured to split the input data into several groups and independently calculate attention for each group;
a vector calculator configured to calculate query, key, and value vectors for each group;
an attention score calculator configured to calculate an attention score through a query and a key and apply a Sparse Mask to calculate relationships between necessary data; and
a calculation result merger configured to merge attention calculation results for each group to generate a final output.

7. The apparatus according to claim 6, wherein the query indicates what data is looking for, the key represents features of the data, and the value indicates an actual value for the key, and
a sparse attention calculation calculates the attention score through the query and the key and applies the sparse mask to calculate relationships only between necessary data.

8. The apparatus according to claim 5, wherein the lightweight GST model part is configured to perform knowledge distillation to transfer knowledge learned from a large model to a small model while maintaining performance of a lightweight model.

9. The apparatus according to claim 8, wherein, in a knowledge distillation process,
a teacher model generates a soft label after recognizing the input data and delivers the soft label to a student model, and
the lightweight student model recognizes the input data and learns the soft label of the teacher model and a ground truth label simultaneously,
wherein learning is performed by combining distillation Loss (KL-divergence) and label loss (cross-entropy).

10. The apparatus according to claim 9, wherein, in a soft label generation step by the teacher model,
the soft label (probability distribution) is generated through softmax based on the input data,
the soft label indicates a probability predicted by the teacher model for each class, which is used to train the student model, and
in a comparison step with the ground truth label, the teacher model is trained through cross-entropy loss with the ground truth label.

11. The apparatus according to claim 8, wherein in a knowledge distillation process,
a student model uses a soft label of a teacher model and a ground truth label for learning to optimize performance, and
for the knowledge distillation, in a comparison step with the soft label, KL-divergence loss is used to train the student model to closely mimic the soft label (prediction probability distribution) of the teacher model, and in this process, knowledge of the teacher model is transferred to the student model.

12. The apparatus according to claim 11, wherein in a back propagation process, the student model is optimized through back propagation based on a total loss which combines two losses (distillation loss and label loss).

13. A method using a lightweight transformer model for human activity recognition in a portable device, the method comprising:
performing a data processing process to process mm Wave data and convert the mmWave data into an input form for a model;
adding location information to input data and normalizing a distribution of the input data;
splitting an input sequence into groups and independently calculating attention for each group;
converting the input data through a feed-forward network (FFN) and enhancing learning expressiveness of the model;
randomly deactivating some neurons to prevent overfitting, maintaining learning information between blocks, and mitigating a gradient vanishing problem; and
normalizing output values of each layer and generating a final feature vector to classify an output class (human activity type) through a fully connected layer.

14. The method according to claim 13, wherein the calculating of the attention comprises:
delivering data to next step with a sequence and location information of the data included;
splitting the input data into several groups and independently calculating attention for each group;
calculating query, key, and value vectors for each group;
calculating an attention score through a query and a key and applying a sparse mask to calculate relationships between necessary data; and
merging attention calculation results for each group to generate a final output.
